(19) United States
(12) Patent Application Publication
(10) Pub. No.: US 2014/0339213 A1
(43) Pub. Date: Nov. 20, 2014

(54) LED Torch

(71) Applicant: Fan Bao, Walnut, CA (US)

(72) Inventor: Fan Bao, Walnut, CA (US)

(21) Appl. No.: 14/451,395

(22) Filed: Aug. 4, 2014

Related U.S. Application Data
(63) Continuation-in-part of application No. 12/925,467, filed on Oct. 21, 2010, now Pat. No. 8,829,395.

(51) Int. Cl. A61M 11/04; A24F 47/00

(52) U.S. Cl. CPC A61M 11/042; A24F 47/008; A61M 2202/0007; A61M 2202/02; A61M 2205/3673
USPC 219/209

(57) ABSTRACT

An LED torch includes a hand-held housing, an LED illumination unit, an inhaler arrangement, and an inhaler control unit. The hand-held housing has an inhaler end, an opposed treatment end, and a treatment cavity. The electric power arrangement includes an electric power source, an electrical-powered control unit, and a heating arrangement provided at the treatment cavity. The LED illumination unit is operatively linked to the electric power source for generating illumination light. The inhaler arrangement includes a tubular inhaler unit retractably coupled at the hand-held housing and being moved between an operation position and a retracted position. The inhaler control unit includes a locker arm for locking up the inhaler unit in the retracted position, and a control button being actuated to disengage the locker arm with the inhaler unit so as to enable the inhaler unit to be moved from the retracted position to the operation position.

### CROSS REFERENCE OF RELATED APPLICATION

This is a Continuation-In-Part application that claims the benefit of priority under 35 U.S.C. §119 to a non-provisional application Ser. No. 12/925,467, filed Oct. 21, 2010.

### BACKGROUND OF THE PRESENT INVENTION

1. Field of Invention

The present invention relates to a vaporizer, and more particular to an LED torch, which is a portable herbal and aromatherapy vaporizer for heating up a treatment element by means of electrical power to extract for inhalation of the ingredient of the treatment element.

2. Description of Related Arts

A vaporizer is a device commonly used for releasing medicinal or psychoactive compounds from plant materials, such as Lavender. For medical or recreational purpose, vaporizing is an alternative to smoking. Rather than burning, a vaporizer heats the plant material to around 200° C. so that the volatile psychoactive and medicinal constituents contained in the plant melt and phase into an aromatic vapor that does not contain the particular matter, such as tars, found in the smoke.

A portable vaporizer is provided with a compact size for enabling the user to carry the vaporizer. Generally speaking, the existing portable vaporizer comprises a hand-held housing containing a fuel chamber, a fuel-powered piezoelectric igniter operatively linked to the fuel chamber, and a mouthpiece holding the substance. Accordingly, once the fuel chamber is filled with combustible fuel, such as butane gas, the piezoelectric igniter is ignited when the combustible fuel is released from the fuel chamber. In other words, the piezoelectric igniter is actuated; the substance will be heated up and extracted for inhalation of the ingredient of the substance via the mouthpiece. However, the existing portable vaporizer has several drawbacks.

The portable vaporizer must be filled by combustible fuel which is highly flammable. In other words, it is unsafe for the user to carry the relatively high amount of combustible fuel. In addition, it is a hassle for the user to refill the combustible fuel once the fuel chamber is empty or near empty. However, the user always has a hard time to find the combustible fuel anywhere for refilling purpose. Otherwise, the user must carry an additional bottle of combustible fuel for refilling the vaporizer in case that the combustible fuel of the vaporizer is used up.

It is appreciated that the volume of the fuel chamber can be increased in order to contain relatively more combustible fuel therein for prolonging the service span of the vaporizer. However, the overall size of the hand-held housing must be corresponding increased to fit the fuel chamber in the hand-held housing. Therefore, the portability of the vaporizer will be substantially reduced. In other words, the vaporizer can be reduced its size to enhance the portability thereof. But, the size of the fuel chamber will be compensated to reduce the volume of combustible fuel therein.

The substance is heated by the vaporizer via the piezoelectric igniter. As to heating, the substance is placed on a metal plate or a hot surface which is heated to release the active constituents. However, the heating temperature of the piezoelectric igniter is hard to be controlled through the combustible fuel, especially to control the heating temperature at a constant temperature. If the heating temperature is too low, the substance cannot be extracted effectively. If the heating temperature is too high, the substance may be burned as a result.

During the operation of the vaporizer, there is always a residual gas remaining in the vaporizer even though the gas valve of the fuel chamber is shut off. Due to the structural configuration of the vaporizer, the residual gas must be gradually released through the piezoelectric igniter. Since the residual gas is highly flammable, the residual gas will be easily burn or ignited, causing fire or combustion. It is worth mentioning that the gas valve and a safety valve must be incorporated with the fuel chamber to control the release of the combustible fuel. Therefore, the manufacturing cost of the vaporizer will be substantially increased by additional components including the gas valve and the safety valve.

In addition, the user will always smell the combustible fuel during the operation of the vaporizer. Even though the vaporizer is switched off, the user will keep inhaling the residual gas for a period of time. It is known that the combustible fuel is unhealthy when it is inhaled.

As it is mentioned above, the safety valve must be incorporated with the fuel chamber for preventing the leakage of the combustible fuel. However, once the safety valve is malfunctioned, the combustible fuel may be accidentally leaked out of the fuel chamber by any unintentional operation such as accidentally pressing the operation button of the vaporizer.

Furthermore, since the vaporizer is powered by combustible fuel, the vaporizer can only provide a single function of inhaling the substance. In other words, the user must carry the vaporizer everywhere for only inhalation of the ingredient of the substance without any other additional function.

### SUMMARY OF THE PRESENT INVENTION

The invention is advantageous in that it provides an LED torch, which is a portable herbal and aromatherapy vaporizer for heating up a treatment element by means of electrical power to extract for inhalation of the ingredient of the treatment element.

Another advantage of the invention is to provide an LED torch, wherein no combustible fuel is used in order to heat up the treatment element so as to provide a safe and convenient way for the user to operate the LED torch.

Another advantage of the invention is to provide an LED torch, which comprises an electric-powered heating element operatively linked to an electric power source such that the heating element will heat up the treatment element by closing the electric circuit between the heating element and the electric power source.

Another advantage of the invention is to provide an LED torch, wherein the user is able to replace the electric power source as the replaceable battery or recharge the electric power source as the rechargeable battery to prolong the service life span of the LED torch instead of refilling the combustible fuel to the conventional vaporizer.

Another advantage of the invention is to provide an LED torch, wherein the mouthpiece and the LED light unit are interchangeable to selectively connect to the electric power source. Therefore, the LED torch of the present invention provides multiple functions of inhaling purpose and illumination purpose.

Another advantage of the invention is to provide an LED torch, wherein the inhaler unit is deposed within the hand-held housing while the LED torch is not in use.

Another advantage of the invention is to provide an LED torch, wherein the inhaler unit can be easily released and withdrawn from the hand-held housing by an inhaler control unit.

Additional advantages and features of the invention will become apparent from the description which follows, and may be realized by means of the instrumentalities and combinations particular point out in the appended claims.

According to the present invention, the foregoing and other objects and advantages are attained by an LED torch which comprises a hand-held housing, an electrical power arrangement, and a multifunctional head unit.

The electric power arrangement comprises an electric power source received in the hand-held housing and an electrical-powered operation head which is electrically linked to the electric power source and is located at an outer wall of the hand-held housing.

The multifunctional head unit comprises an inhaler unit and an LED illumination unit detachably and interchangeably coupled with the hand-held housing to operatively link to the operation head, wherein the inhaler unit has a mouthpiece and a treatment cavity for receiving a treatment element therein. When the inhaler unit is operatively coupled with the operation head of the electric power arrangement, the operation head forms a heating head for heating the treatment element in the treatment cavity so as to extract the treatment element for inhalation of an ingredient thereof via the mouthpiece. When the LED illumination unit is operatively coupled with the operation head of the electric power arrangement, the operation head forms an electrical adapter to electrically link the LED illumination unit with the electric power source for generating illumination light.

According to the present invention, the foregoing and other objects and advantages are attained by an LED torch which comprises:

a hand-held housing having an elongated structure and defining an inhaler end, an opposed treatment end, and a treatment cavity formed at the treatment end for receiving a treatment element in the treatment cavity;

an electric power arrangement which comprises an electric power source received in the hand-held housing, an electrical-powered control unit which is electrically linked to the electric power source, and a heating arrangement provided at the treatment cavity for heating and extracting the treatment element therein;

an LED illumination unit provided at the treatment end of the hand-held housing and operatively linked to the electric power source for generating illumination light;

an inhaler arrangement which comprises a tubular inhaler unit retractably coupled at the hand-held housing and being moved between an operation position and a retracted position, wherein in the operation position, the inhaler unit is longitudinally extended out of the inhaler end of the hand-held housing to communicate with the treatment cavity for inhalation of an ingredient of the treatment element, wherein in the retracted position, the inhaler unit is longitudinally received in the hand-held housing; and

an inhaler control unit which comprises a locker arm movably provided at the held-hand housing to lock up the inhaler unit in the retracted position, and a control button being actuated to disengage the locker arm with the inhaler unit so as to enable the inhaler unit to be moved from the retracted position to the operation position.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a sectional view of an LED torch according to a preferred embodiment of the present invention, illustrating an inhaler unit being detachably coupled with the hand-held housing.

FIG. 2 is a sectional view of the LED torch according to the above preferred embodiment of the present invention, illustrating an LED illumination being detachably coupled with the hand-held housing.

FIG. 3 is a side view of the hand-held housing of the LED torch according to the above preferred embodiment of the present invention.

FIG. 4 is a front view of the hand-held housing of the LED torch according to the above preferred embodiment of the present invention.

FIG. 5 illustrates a clearance between the heater head socket and the inhaler unit according to the above preferred embodiment of the present invention.

FIG. 6 is a perspective view of an LED torch according to a second preferred embodiment of the present invention.

FIG. 7A is a perspective view of the LED torch according to the above preferred embodiment of the present invention, illustrating an open situation of the treatment cavity.

FIG. 7B is a perspective view of the LED torch according to the above preferred embodiment of the present invention, illustrating an LED light arranged on a bottom face of the hand-held housing.

FIG. 8 is a cross sectional view of the LED torch according to the above preferred embodiment of the present invention.

FIG. 9A is a perspective view of the LED torch according to the above preferred embodiment of the present invention, illustrating an operation position of the inhaler unit.

FIG. 9B is a perspective view of the LED torch according to the above preferred embodiment of the present invention, illustrating a retracted position of the inhaler unit.

FIG. 10A illustrates an alternative mode of the inhaler control unit of the LED torch according to the above preferred embodiment of the present invention, illustrating an operation position of the inhaler unit.

FIG. 10B illustrates the alternative mode of the inhaler control unit of the LED torch according to the above preferred embodiment of the present invention, illustrating a retracted position of the inhaler unit.

### DETAILED DESCRIPTION OF THE PREFERRED EMBODIMENT

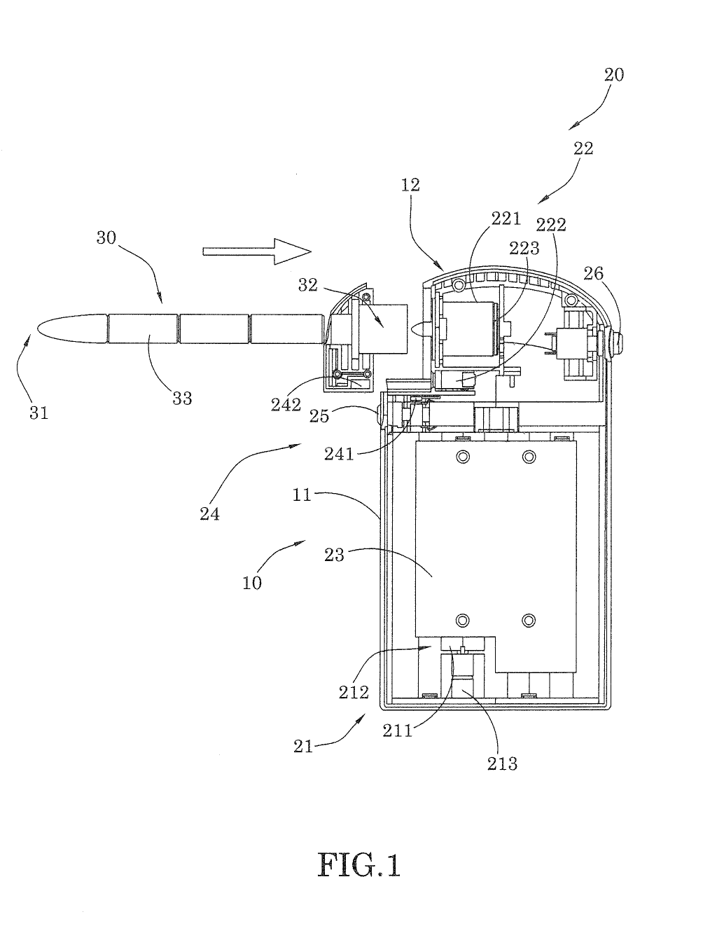

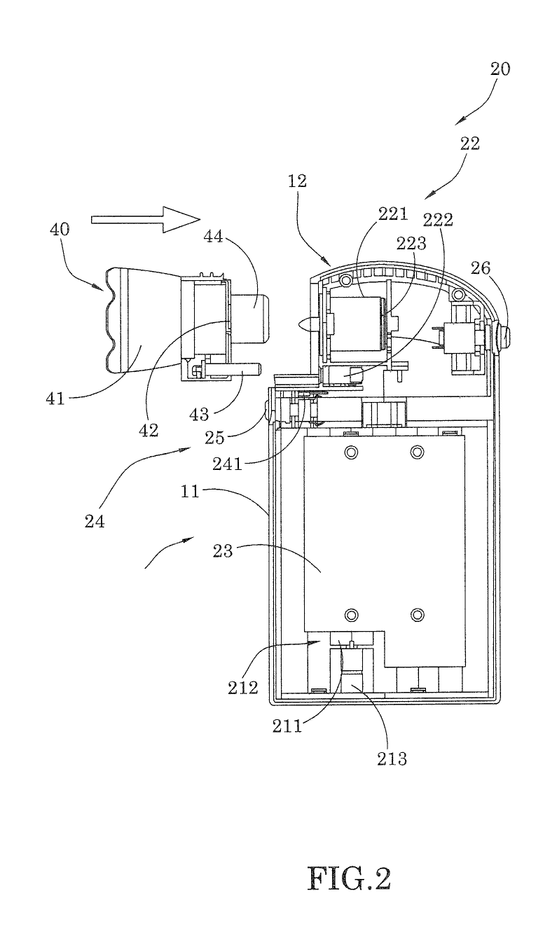

Referring to FIGS. 1 and 2 of the drawings, an LED torch according to a preferred embodiment of the present invention is illustrated, wherein the LED torch, which is embodied as a portable herbal and aromatherapy vaporizer, comprises a hand-held housing 10, an electrical power arrangement 20, and a multifunctional head unit.

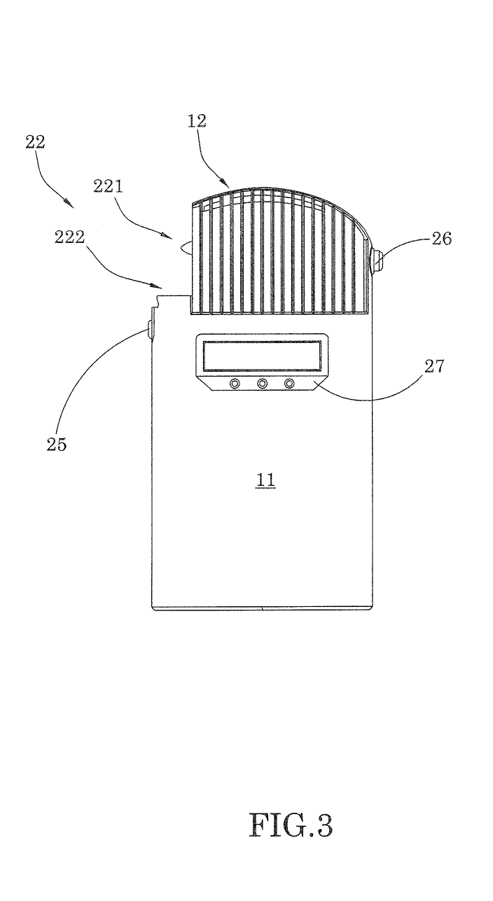

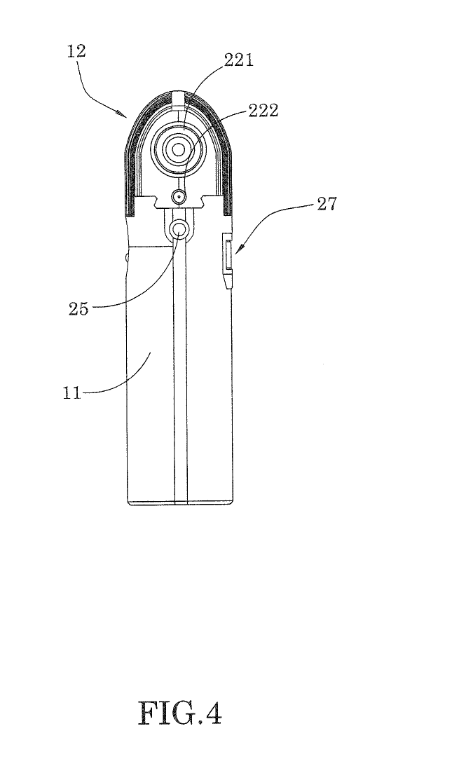

As shown in FIGS. 3 and 4, the hand-held housing 10 has a hand size adapted for being held of the hand of the user, wherein the hand-held housing 10 has an outer wall 11 defining a top face, a bottom face, two side faces, a front face, and a rear face.

The electric power arrangement 20 comprises an electric power source 21 received in the hand-held housing 10 and an electrical-powered operation head 22 which is electrically linked to the electric power source 21 and is located at the outer wall 11 of the hand-held housing 10. In particular, the operation head 22 is located at the front face of the hand-held housing 10 at the upper portion thereof.

The multifunctional head unit comprises a plurality of functional units being interchangeably coupled with the hand-held housing 10 to operatively link to the electric power arrangement 20 for providing different functions.

In particular, the multifunctional head unit comprises an inhaler unit 30 and an LED illumination unit 40 detachably and interchangeably coupled with the hand-held housing 10 to operatively link to the operation head 22.

As shown in FIG. 1, the inhaler unit 30 has a mouthpiece 31 and a treatment cavity 32 for receiving a treatment element therein. According to the preferred embodiment, the inhaler unit 30 comprises a tubular inhaling guider 33 longitudinally supported by the hand-held housing 10, wherein the inhaling guider 33 has a free end defining the mouthpiece 31 thereat and a detachable end defining the treatment cavity 32 thereat. The detachable end of the inhaler unit 30 is detachably coupled with the operation head 22 to detachably support the inhaler unit 30 at the hand-held housing 10.

When the inhaler unit 30 is operatively coupled with the operation head 22 of the electric power arrangement 20, the operation head 22 forms a heating head for heating the treatment element in the treatment cavity 32 so as to extract the treatment element for inhalation of an ingredient thereof via the mouthpiece 31.

As shown in FIG. 2, the LED illumination unit 40 comprises an LED light casing 41 detachably coupling with the operation head 22 and an LED light source 42 received in the LED light casing 41. When the LED illumination unit 40 is operatively coupled with the operation head 22 of the electric power arrangement 20, the operation head 22 forms an electrical adapter to electrically link the LED illumination unit 40 with the electric power source 42 for generating illumination light.

According to the preferred embodiment, the electric power source 21 comprises one or more rechargeable batteries 211 received in a battery compartment 212 in the hand-held housing 10 and a charging outlet 213 provided at the outer wall 11 of the hand-held housing 10 at the bottom face thereof for charging the rechargeable batteries 211 via AC power. Alternatively, the electric power source 21 can be the replaceable batteries that the rechargeable batteries 212 are replaced by the replaceable batteries in the battery compartment 213.

As shown in FIGS. 1 and 2, the electric power arrangement 20 further comprises a control circuit 23 controllably linked between the operation head 22 and the electric power source 21 and an automatic switch 24 which is provided at the operation head 22 and is controlled by the control circuit 23 to selectively switch an operation between the inhaler unit 30 and the LED illumination unit 40.

According to the preferred embodiment, the control circuit 23 is a circuit supported in the hand-held housing 10, wherein the control circuit 23 has a first control circuit configuration to control the operation of the operation head 22 as the heating head for the inhaler unit 30 and a second control circuit configuration to control the operation of the operation head 22 as the electric adapter for the LED illumination unit 40. Accordingly, the first control circuit configuration is a heating control configuration while the second control circuit configuration is a light control configuration.

The control circuit 23 further has a charging protection circuit configuration linked to the electric power source 21 for preventing the short circuit of the control circuit 23 and the over-charging of the electric power source 21. It is worth mentioning that the electric power source 21 can be re-charged over 800 times.

In addition, the electric power arrangement 20 further comprises a display panel 27 provided at one of the side faces of the outer wall 11 of the hand-held housing 10 as shown in FIG. 3, wherein the display panel 27 has a plurality of light indicators electrically linked to the control circuit 23 for indicating the status of the LED torch. For example, one of the light indicators will be flashed when the electric power source 21 is charging to indicate the charging status of the electric power source 21. One of the light indicators will be flashed to indicate the electrical power level of the electric power source 21. One of the light indicators will be flashed to indicate the operation status, such as on-and-off status of the electric power arrangement 20.

The automatic switch 24 is embodied as an auto detector for detecting the connection of the operation head 22 with respect to one of the inhaler unit 30 and the LED illumination unit 40. In particular, the automatic switch 24 is operatively linked to the first and second circuit configurations of the control circuit 23, such that when the inhaler unit 30 is coupled at the operation head 22, the first control circuit configuration of the control circuit 23 is activated while the second control circuit configuration of the control circuit 23 is deactivated. Likewise, when the LED illumination unit 40 is coupled at the operation head 22, the first control circuit configuration of the control circuit 23 is deactivated while the second control circuit configuration of the control circuit 23 is activated.

Preferably, the automatic switch 24 comprises a magnetic sensor switch 241 provided at the operation head 22 and a magnetic element 242 only provided at the inhaler unit 30 at the detachable end thereof. Therefore, when the inhaler unit 30 is detachably coupled with the operation head 22, the magnetic sensor switch 241 is switched on by detecting a presence of magnetic field via the magnetic element 242, such that the control circuit 23 automatically configures the operation head 22 as the heating head for heating up the treatment element by activating the first control circuit configuration only.

When the LED illumination unit 40 is detachably coupled with the operation head 22, the magnetic sensor switch 241 is switched off by detecting no magnetic field at the LED illumination unit 40, such that the control circuit 23 automatically configures the operation head 22 as the electrical adapter to electrically connect with the LED illumination unit 40 by activating the second control circuit configuration only.

It is appreciated that other detecting switches can be used for detecting the connection of the operation head 22 with respect to one of the inhaler unit 30 and the LED illumination unit 40 in order to activate one of the first and second control circuit configurations.

According to the preferred embodiment, the operation head 22 comprises a heater head socket 221 detachably coupling with the inhaler unit 30 and a light head socket 222 which is positioned adjacent to the heater head socket 221 to detachably couple with the LED illumination unit 40. Accordingly, the heater head socket 221 and the light head socket 222 are operatively linked to the first and second control circuit configurations of the control circuit 23 respectively.

As shown in FIG. 1, the heater head socket 221 is positioned at the front face of the outer wall 11 of the hand-held housing 10, wherein a heating element 223 is controllably linked with the control circuit 23 and is positioned behind the heater head socket 221 for heat generation. Accordingly, the heating element 223 can be ceramic, silicon, or metal adapted for heat generation. It is worth mentioning that the heating temperature of the heating element 223 can be precisely controlled by the control circuit 23 by controlling the current passing therethrough. It is appreciated that using other electronic components such as capacitors, resistors, or the like can precisely control the heating temperature of the heating element 223.

The heater head socket 221 is made of heat conductive material and is arranged in such a manner that when the heating element 223 is electrically activated by the control circuit 23, i.e. the activation of the first control circuit configuration, the heater head socket 221 will form the heating head for heat generation. The heating temperature of the heating element 223 can be adjusted as high as 200° C. while the heating temperature of the heater head socket 221 can be adjusted as high as 50 to 60° C. Therefore, when the detachable end of the inhaler unit 30 is coupled at the heater head socket 221, the heat from the heater head socket 221 will transmit to the treatment cavity 32 to as to extract the treatment element therein for inhalation of an ingredient thereof via the mouthpiece 31. It is worth mentioning that the treatment cavity 32 is formed at the detachable end of the inhaler unit 30 and is made of heat conductive material for effectively conducting heat from the heater head socket 221.

Accordingly, the LED torch of the present invention further comprises a PTC temperature sensor operatively linked to the control circuit 23 to detect the heating temperature of the heating element 223, wherein the PTC temperature sensor is adapted for avoiding the overheating of the heater head socket 221 and burning the treatment element within the treatment cavity 32 by the heat from the heater head socket 221.

According to the preferred embodiment, the hand-held housing 11 has a plurality of air vents 12 provided at the outer side thereof adjacent to the operation head 22 for heat dissipation. In particular, the air vents 12 are a plurality of through slots formed at the top portion of the hand-held housing 11 adjacent to the location of the heater head socket 221 for heat dissipation.

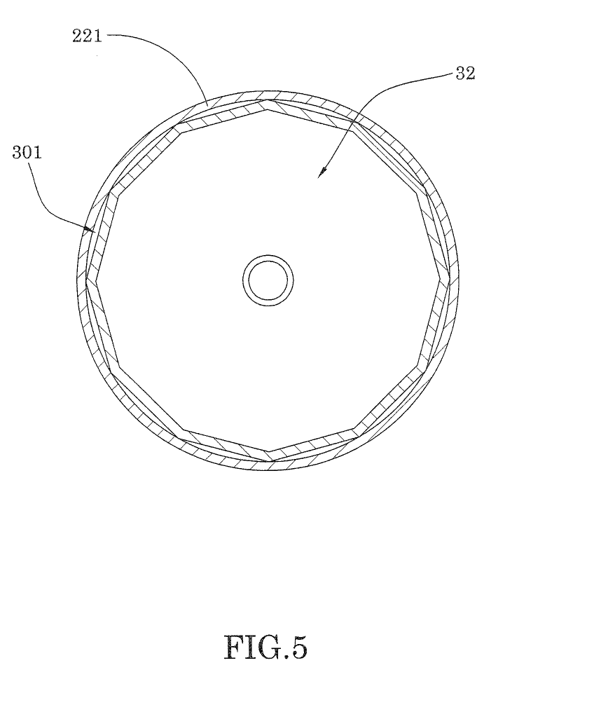

As shown in FIG. 5, the heater head socket 221 has a circular cross section while the detachable end of the inhaler unit 30, i.e. the treatment cavity 32, has a polygonal cross section. Therefore, when the detachable end of the inhaler unit 30 is detachably coupled with the heater head socket 221, a clearance 301 is formed between an inner surface of the heater head socket 221 and an outer surface of the detachable end of the inhaler unit 30 for air circulation. In other words, during the inhalation of the ingredient of the treatment element via the mouthpiece 31, air (oxygen) can pass through the clearance 301 into the treatment cavity 32 for enhancing the extraction of the treatment element.

As shown in FIG. 2, the light head socket 222 is positioned at the front face of the outer wall 11 of the hand-held housing 10 at the position below the heater head socket 221, wherein the light head socket 222 forms the electrical adapter to electrically link the LED illumination unit 40 with the electric power source 42. The LED illumination unit 40 further comprises an electrical terminal 43 rearwardly extended from the LED light casing 41 and electrically linked to the LED light source 42, wherein the electrical terminal 43 is detachably coupled with the light head socket 222 to electrically link the LED light source 42 with the electric power arrangement 20. The LED illumination unit 40 further comprises a retention head 44 rearwardly extended from the LED light casing 41 to align with the heater head socket 221, wherein when the electrical terminal 43 is detachably coupled with the light head socket 222, the retention head 44 is detachably coupled with the heater head socket 221 to retain the LED illumination unit 40 in position. It is worth mentioning that when the electrical terminal 43 is detachably coupled with the light head socket 222, the first control circuit configuration of the control circuit 23 is deactivated. Therefore, the heater head socket 221 will not generate heat. Thus, the heater head socket 221 forms a retention member to detachably couple with the retention head 44 to hold the LED illumination unit 40 in position. In addition, when the detachable end of the inhaler unit 30 is detachably coupled with the heater head socket 221, the bottom portion of the detachable end of the inhaler unit 30 will cover up the light head socket 222.

As shown in FIGS. 1 to 4, the electric power arrangement 20 further comprises an operation actuator 25 and a safety actuator 26 spacedly provided at the outer wall 11 of the hand-held housing 10 to operatively linked to the control circuit 23. Accordingly, the operation actuator 25 and the safety actuator 26 are provided at the front and rear faces of the outer wall 11 of the hand-held housing 10. The operation actuator 25 and the safety actuator 26 must be actuated at the same time in order to start operating the electric power arrangement.

According the operation of the LED torch of the present invention is illustrated as follows.

For connection of the inhaler unit 30, the user is able to fill the treatment element in the treatment cavity 32. The detachable end of the inhaler unit 30 can be detachably coupled with the heater head socket 221 of the operation head 22. Then, the user must press the operation actuator 25 and the safety actuator 26 at the same time for a predetermined time, such as 2 seconds, in order to switch on the electric power arrangement 20 at the standby mode. Once the detachable end of the inhaler unit 30 is detachably coupled with the heater head socket 221, the operation actuator 25 can be pressed to start the heat generation from the heater head socket 221. It is worth mentioning that when the inhaler unit 30 is detachably coupled with the heater head socket 221, the magnetic sensor switch 241 is switched on by detecting a presence of magnetic field via the magnetic element 242, such that the control circuit 23 automatically configures the heater head socket 221 as the heating head for heating up the treatment element by activating the first control circuit configuration only. Accordingly, the heating temperature will reach 200° C. for about one minute. In addition, the fully charged electrical power from the electric power source 21 can provide 5 hours of heating operation of the heater head socket 221 for the inhaler unit 30. In order to stop the operation of the heater head socket 221, the user is able to press the operation actuator 25 again so as to deactivate the first control circuit configuration for stop generating heat. It is worth mentioning that the control circuit 23 will also stop the operation of the heater head socket 221 after a predetermined of continuous using time, such as 20 minutes, for preventing the overheating of the operation head 22. The user is able to press the safety actuator 26 once in order to switch off the electric power arrangement 20 of the LED torch from the standby mode to an off mode.

It is worth mentioning that the heat from the operation head 22 is powered by the electric power source 21 instead of conventional piezoelectric igniter powered by combustible fuel, i.e. butane gas. Therefore, the user does not have to refill the butane gas once the butane gas is used up or have to carry additional butane gas bottle for refilling purpose. In other words, no leakage of the combustible fuel will be occurred in the present invention. In particular, once the operation actuator 25 is pressed twice to deactivate the first control circuit configuration for stop generating heat, no residual butane gas is left in the hand-held housing 10, such that the user will not inhale any butane gas before and after the operation of the LED torch of the present invention. On the other hand, the LED torch has no air pollution due to the absence of the butane gas. When the electrical power level of the electric power source 21 is low, the user is able to recharge the rechargeable battery as the electric power source 21 by electrically plugging the electric power source 21 to the AC outlet via the charging outlet 213 or replacing the replaceable battery as the electric power source 21.

For connection of the LED illumination unit 40, the user is able to detachably couple the LED illumination unit 40 with the light head socket 222 of the operation head 22. Then, the user must press the operation actuator 25 and the safety actuator 26 at the same time for a predetermined time, such as 2 seconds, in order to switch on the electric power arrangement 20 at the standby mode. Once the LED illumination unit 40 is detachably coupled with the light head socket 222, the operation actuator 25 can be pressed to switch on the LED light source 42. The fully charged electrical power from the electric power source 21 can provide 15 hours of illumination operation for the LED illumination unit 40. It is worth mentioning that the LED illumination unit 40 will generate light for illumination when the operation actuator 25 is pressed once. The illumination unit 40 will generate light with flash manner when the operation actuator 25 is pressed twice. The illumination 40 will be switched off when the operation actuator 25 is pressed at the third time. Likewise, the user is able to press the safety actuator 26 once in order to switch off the electric power arrangement 20 of the LED torch from the standby mode to the off mode.

It is appreciated that the LED illumination unit 40 can be replaced by other electrical devices powered by the electric power arrangement 20.

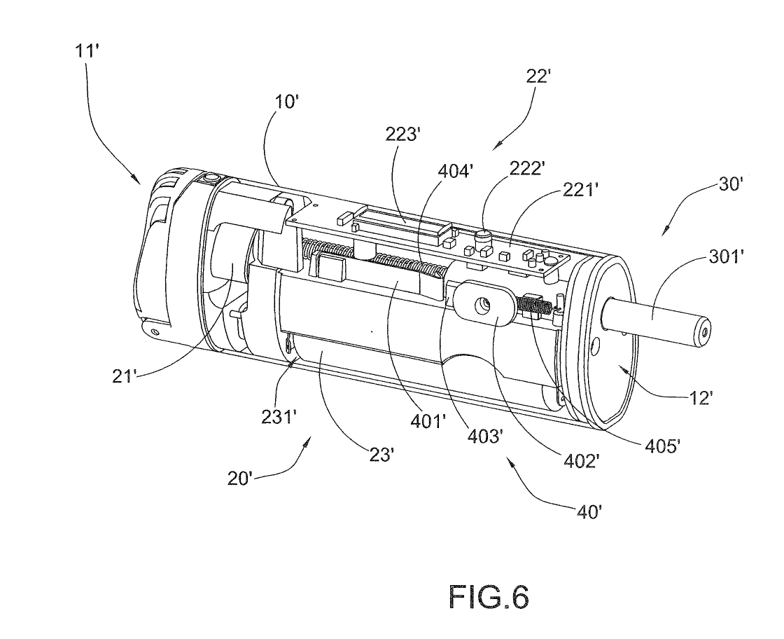

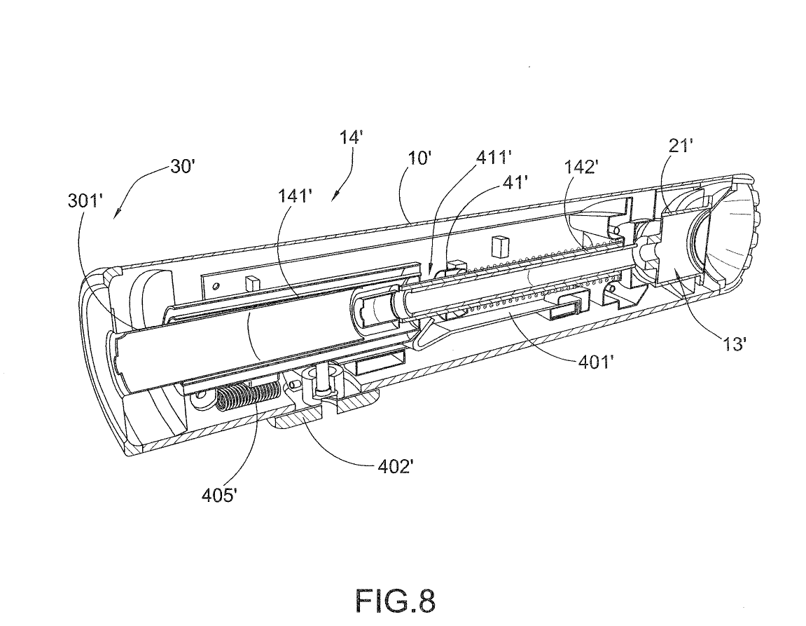

Referring to FIG. 6 to FIG. 8 of the drawings, an LED torch according to a second preferred embodiment of the present invention is illustrated, wherein the LED torch comprises a hand-held housing 10′ having an elongated structure, an electrical power arrangement 20′, an LED illumination unit 51′, and an inhaler arrangement 30′.

Accordingly, the hand-held housing 10′ has a hand size adapted for being held of the hand of the user, wherein the hand-held housing 10′ has an outer wall defining a top face, a bottom face, two side faces, a front face, and a rear face. The hand-held housing 10′ comprises an inhaler end 11′, a treatment end 12′, a treatment cavity 13′ formed at the treatment end 12′, and a tubular guiding member 14′ extended from the treatment cavity 13′ to the inhaler end 11′.

The guiding member 14′ comprises an inner pipe 142′ extended from the treatment cavity 13′ and an intermediate pipe 141′ coaxially extended from the inner pipe 142′, wherein a diameter of the intermediate pipe 141′ is larger than a diameter of the inner pipe 142′.

The electrical power arrangement 20′ comprises an electric power source 23′ received in the hand-held housing 10′, an electrical-powered control unit 22′ which is electrically linked to the electric power source 23′, and a heating arrangement 21′ provided at the treatment cavity 13′ for heating and extracting the treatment element therein, wherein the electrical-powered control unit 22′ comprises a control circuit 221′ supported in the hand-held housing 10′ for controllably linked with the electric power source 23′, and an inhaler switch 222′ operatively linked to the control circuit 221′ to selectively activate the heating arrangement 21′. In addition, inhaler switch 222′ is able to control an “Operation” and “Stop” status of the LED torch.

In other words, while the inhaler switch 222′ is activated, the electric power source 23′ will generate the electric power and deliver it to the heating arrangement 21′, such that the heating arrangement 21′ will transfer the electric power to a heating source. In addition, the inhaler switch 222′ is able to activate again to stop the electric power source 23′ generating the electric power, such that the heating arrangement 21′ will stop generate the heating source. Preferably, the inhaler switch 222′ is a press button.

It is worth mentioning that the heating arrangement 21′ is activated by the inhaler switch 222′ to generate two different kinds of heating level, which is a first level heating and a second level heating. The second level heating can provide a stronger power source than the first level heating to the treatment cavity 13′, such that the user can selectively chose a suitable heating level as their needs.

Moreover, the control circuit 221′ comprises a LCD screen 223′ electrically connected with the control circuit 223′ to control the information indicated on the LCD screen 205′. While the LED torch is in the “Operation” status, the “On” image will be displayed on the LCD screen 223′. In other words, while the LED torch is on “Stop” status, the LCD screen 223′ will display an “Off” image. And, the LCD screen 223′ not only can display a lifespan of the power of the LED torch, but also can display the heating level thereof, such that the power level of the LED torch can be displayed on the LCD screen 223′ to facilitate the user to charge the LED torch.

Furthermore, the LED torch further comprises an LED illumination 51′ provided a the treatment end 11′ of the hand-held housing 10′ and operatively linked to the electric power source 23′ for generating illumination light, wherein the LED illumination unit 51′ is electrically connected to the control circuit 221′, so the LED illumination unit 51′ is selectively controlled by the inhaler switch 222′. Therefore, the inhaler switch 222′ is able to turn on or turn off the LED illumination unit 51′.

It is worth to mentioning that the inhaler switch 222′ is held on to turn on the “Operation” status of the LED torch, and is held on again to turn on “Stop” status of the LED torch. Continuously, the inhaler switch 22′ is able to press once time to generate the first level heating, and press twice times to generate the second level heating, and continuously to press three times to stop all the function in a standby mode. In addition, the inhaler unit 22′ is continuous to press twice times to turn on the LED illumination unit 51′, and press twice times again to turn off the LED illumination unit 51′. In other words, the inhaler switch 222′ is held to turn on the unit of the present invention, and held again to turn off the unit. Pressing the inhaler switch 222′ one time is the first level heating, pressing a second time the inhaler switch 222′ is the second level heating. Consecutively pressing the inhaler switch 222′ twice turns on and off the LED lights, pressing three times the inhaler switch 222′ puts the unit in the standby mode.

According to the preferred embodiment, the electric power source 23′ comprises one or more rechargeable batteries received in a battery compartment 231′ in the hand-held housing 10′. Alternatively, the rechargeable batteries can be replaced to replaceable batteries.

The inhaler arrangement 30′ comprises a tubular inhaler unit 301′ retractably coupled at the hand-held housing and 10′ being moved between an operation position and a retracted position, wherein the inhaler unit 301′ is coaxially and slidably coupled within the intermediate pipe 141′. In the operation position, the inhaler unit 301′ is longitudinally extended out of the inhaler end 12′ of the hand-held housing 10′ to communicate with the treatment cavity 13′ for inhalation of an ingredient of the treatment element. In the retracted position, the inhaler unit 301′ is longitudinally received in the hand-held housing 10′.

Accordingly, the inhaler unit 301′ is supported within the hand-held housing 10′ between said electrical-powered control unit 22′ and the electric power source 23′, such that the electrical-powered control unit 22′ is supported above the inhaler unit 301′ while the electric power source 23′ is supported below the inhaler unit 301′.

The inhaler control unit 40′ comprises a locker arm 401′ movably provided at the hand-held housing 10′ to lock up the inhaler unit 301′ in the retracted position, and a control button 402′ being actuated to disengage the locker arm 401′ with the inhaler unit 301′ so as to enable the inhaler unit 301′ to be moved from the retracted position to the operation position.

It is worth mentioning that the locker arm 401′, which is made of bendable material, has an affixing end affixed at said hand-held housing 10′ and a hooking end engaging with the inhaler unit 301′ to lock up the inhaler unit 301′ in the retracted position, such that when the control button 402′ is moved to bend the hooking end of the locker arm 401′ outwardly, the hooking end of the locker arm 401′ is disengaged with the inhaler unit 301′ to enable the inhaler unit 301′ to be moved from the retracted position to the operation position.

According to the preferred embodiment of the present invention, the inhaler control unit 40′ further comprises a control slider 41′, having a locking groove 411′, coupled at an inner end of the inhaler unit 301′ and slidably coupled at the guiding member 14′, wherein the hooking end of the locking arm 401′ is engaged with the locking groove 411′ of the control slider 41′ to lock up the inhaler unit 301′ in the retracted position. In addition, the control slider 41′ is movably mounted on an outer surface of the inner pipe 143′ of the guiding member 14′. In other words, the inhaler unit 301′ can be longitudinally moving along inner surface of the intermediate pipe 141′ of the guiding member 14′ via the movement of the control slider 41′.

The inhaler control unit 40′ further comprises a slider actuator 403′ extended from the control button 402′ to align with the hooking end of the locker arm 401′, and a resilient element 404′ coaxially mounted at the inner pipe 142′ of the guiding member 14′ for applying a pushing force against the inhaler unit 301′ so as to push the inhaler unit 301′ from the retracted position to the operation position, such that when the control button 402′ is aligned moved toward the locker arm 401′, the hooking end of the locker arm 401′ is pushed by the slider actuator 403′ so as to bend the locker arm 401′ outwardly to disengage with the inhaler unit 301′.

In addition, since the control button 402′ is operatively linked with the slider actuator 403′, the movement of the control button 402′ and the slider actuator 403′ is a linkage movement. In other words, the movement of the control button 402′ can control the movement of the slider actuator 403′, so while the control button 402′ moves towards the resilient element 404′, the slider actuator 403′ moves towards the resilient element 404′ accordingly.

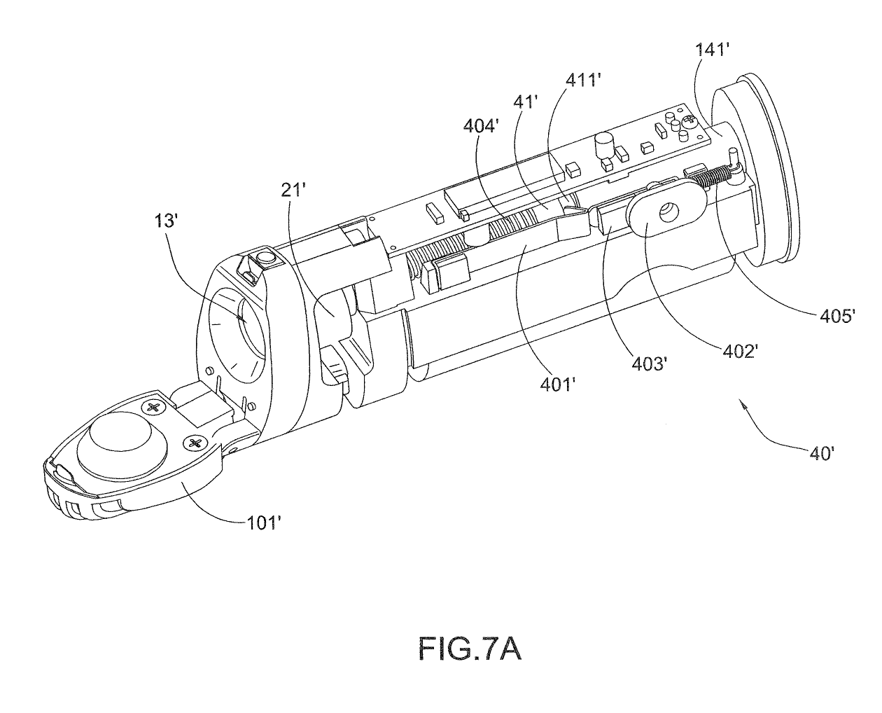

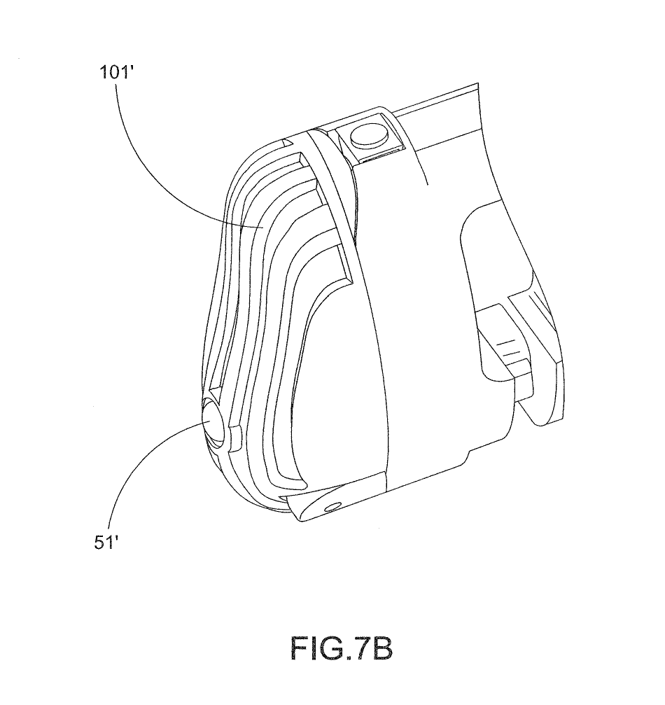

As shown in FIG. 7A to 7B, the hand-held housing 10′ further comprises a enclosure cap 101′ pivotally coupled at the treatment end 12′ of the hand-held housing 10′ to sealedly enclose the treatment cavity 13′. Since one end of the inner pipe 142′ is operatively linked with the treatment cavity 13′ and the other end of the inner pipe 142′ is operatively linked with the inhaler unit 301′, so the extracted treatment element only can be delivered from the treatment cavity 13′ to the inhaler unit 301′ via the inner pipe 142′ of the guiding member 14′ to the in haler unit 301′ for inhalation.

Accordingly, the inhaler switch 222′ of the electrical-power control unit 22′ is electrically linking with the electric power source 23′ of the electric power arrangement 20′. Therefore, in the “Operation” status, the control circuit 221′ is activated by the inhaler switch 222′ to drive the electric power source 23′ to generate the electric power to the heating arrangement 21′ to generate the heating source to the treatment cavity 13′ for heating the treatment element within the treatment cavity 13′ and form an extracted treatment element for inhalation of an ingredient thereof.

In the “Stop” status, the control circuit 221′ is activated by the inhaler switch 222′ to atop generating the electric power from the electric power source 23′ to the heating arrangement 21′ to generate the heating source to the treatment cavity 13′, so no extracted treatment element will be generated from the treatment element in the treatment cavity 13′.

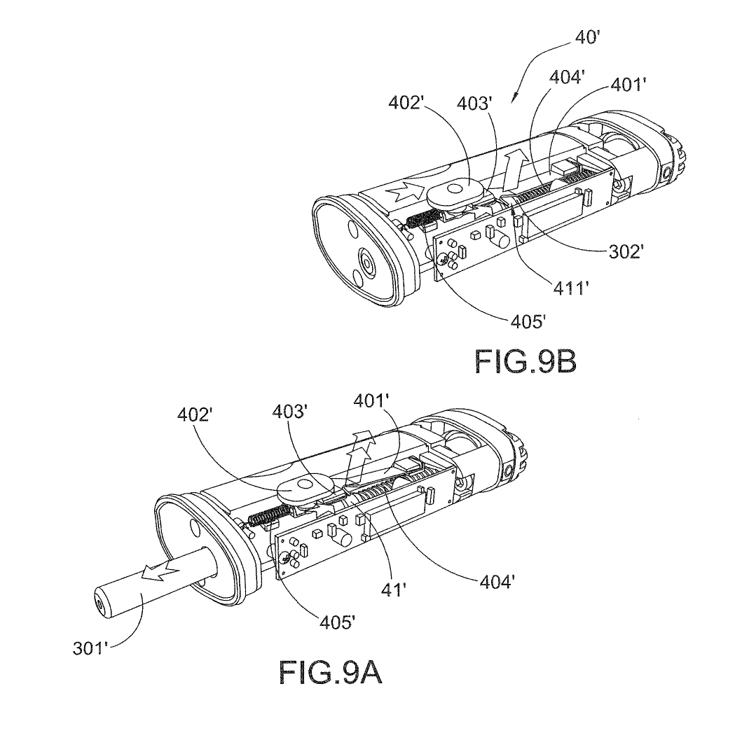

Referring to FIG. 9A of the drawings, in the operation position, the control button 402′ of the inhaler control unit 40′ can be pushed towards the hooking end of the locking arm 401′ so as to release the inhaler unit 301′ from the retracted position. In other words, the slidable control slider 41′ is moving towards the hook end of the locking arm 401′ to bend the hook end of the locking arm 401′, such that the hook end of the locking arm 401′ is disengaged with the locking groove 411′ of the control slider 41′, and in such manner that no squeezed force will be exerted on the resilient element 404′, so the resilient element 404′ will reinstate to its original shape so as to push the control slider 41′ for pushing the inhaler unit 301′ out of the intermediate pipe 141′ of the guiding member 14′.

Referring to FIG. 9B of the drawings, in order to achieve the retracted position, the inhaler unit 301′ is pushed towards the control slider 41′ along intermediate pipe 141′ of the guiding member 14′, such that the hooking end of the locking arm 401′ will be engaged with the locking groove 411′ of the control slider 41′ to push the control slider 41′ to squeeze the resilient element 404′ for locking the inhaler unit 301′ in the retracted position.

It is worth mentioning that the inhaler control unit 40′ further comprises a retraction spring 405′ coupled between the slider actuator 403′ and an inner wall of the top face of the hand-held housing 10′ for pulling the control button 402′ after the control button 402′ is pushed toward the locking arm 401′. While the control button 402′ is pushed towards the hook end of the locking arm 401′, the retraction spring 405′ is stretched along the movement of the control button 402′. And, when the hook end of the locking arm 401′ is bent to disengage with the locking groove 411′ of the control slider 41′, a resilience force of the hook end of the locking arm 401′ is generated towards the slider actuator 403′ and activate the slider actuator 403′ moving towards the retraction spring 405′, such that the retraction spring 405′ will reinstate to its original position via the movement of the slider actuator 403′, so as to activate the control button 402′ returning to its original position.

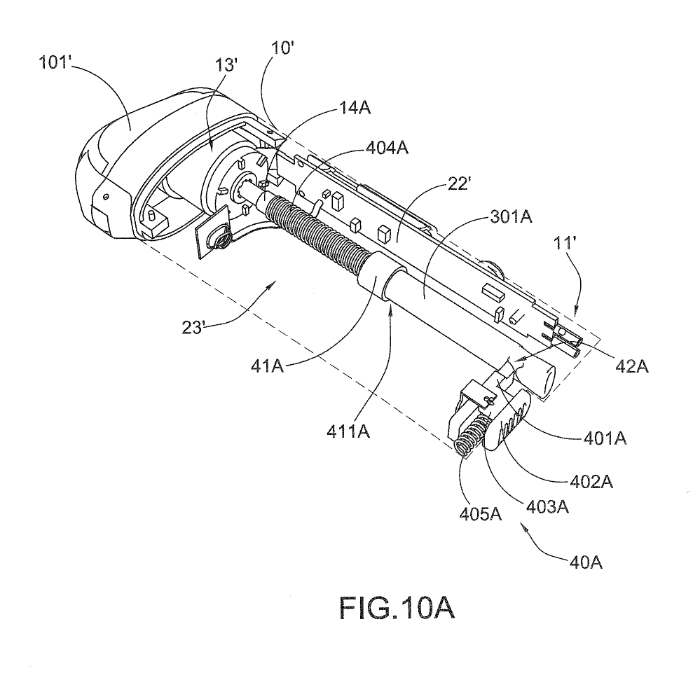

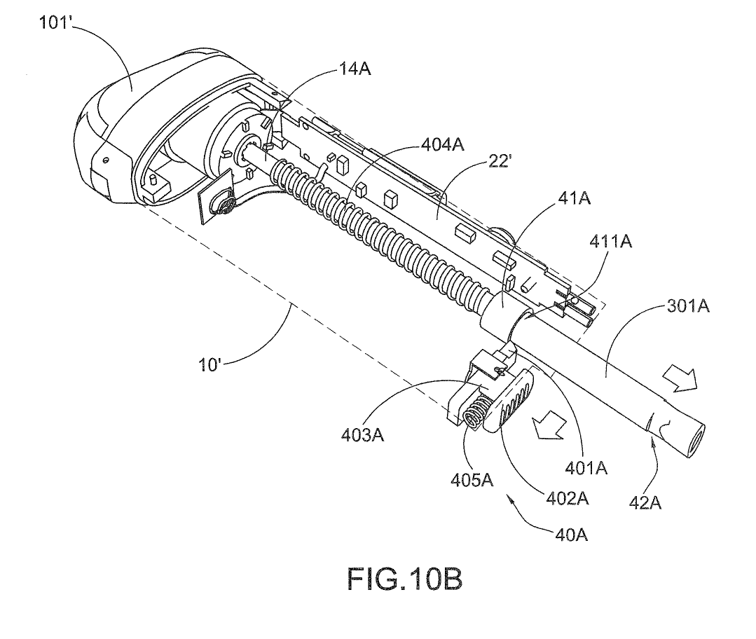

FIGS. 10A and 10B illustrate an alternative mode of the LED torch, which is a modification of the LED torch of the second embodiment. Accordingly, the tubular guiding member 14A of the hand-held housing 10 is directly extended from the treatment cavity 13′ to the inhaler end 11′.

Accordingly, the inhaler arrangement 30A comprises a tubular inhaler unit 301A retractably coupled at the hand-held housing and 10′ being moved between an operation position and a retracted position, wherein the inhaler unit 301A is coaxially and slidably coupled at the guiding member 14A. In the operation position, the inhaler unit 301A is longitudinally extended out of the inhaler end 12′ of the hand-held housing 10′ to communicate with the treatment cavity 13′ for inhalation of an ingredient of the treatment element. In the retracted position, the inhaler unit 301A is longitudinally received in the hand-held housing 10′. In particular, the inhaler unit 301A is supported within the hand-held housing 10′ between said electrical-powered control unit 22′ and the electric power source 23′, such that the electrical-powered control unit 22′ is supported above the inhaler unit 301A while the electric power source 23′ is supported below the inhaler unit 301A. In addition, the outer diameter of the guiding member 14A is slightly smaller than the inner diameter of the inhaler unit 301A, such that the inhaler unit 301A is adapted to slide along the guiding member 14A.

The inhaler control unit 40A comprises a locker arm 401A movably provided at the hand-held housing 10′ to lock up the inhaler unit 301A in the retracted position, and a control button 402A being actuated to disengage the locker arm 401A with the inhaler unit 301A so as to enable the inhaler unit 301A to be moved from the retracted position to the operation position.

The locker arm 401A, which is made of rigid material, has a locking end engaging with the inhaler unit 301A to lock up the inhaler unit 301A in the retracted position, such that when the control button 402A is moved to drive the locking end of the locker arm 401A away from the inhaler unit 301A, the locking end of the locker arm 401A is disengaged with the inhaler unit 301A to enable the inhaler unit 301A to be moved from the retracted position to the operation position.

The inhaler control unit 40A further has a locking shoulder 42A integrally formed at the inhaler unit 301′. In particular, the locking shoulder 42A is indently formed at the surrounding wall of the inhaler unit 301A, such that the locking shoulder 42A forms a chamfered edge at the surrounding wall of the inhaler unit 301A. Accordingly, the locking end of the locking arm 401A is engaged with the locking shoulder 42A to lock up the inhaler unit 301A in the retracted position.

The inhaler control unit 40A further comprises a control slider 41A, having a locking groove 411A, coupled at the inner end of the inhaler unit 301A and slidably coupled at the guiding member 14A, wherein the locking end of the locking arm 401A is engaged with the locking groove 411A of the control slider 41A to retain the inhaler unit 301A in the operation position. In addition, the control slider 41A is configured to have a ring shape coaxially coupled at the inner end of the inhaler unit 301A, such that when the inhaler unit 301A is slid along the guiding member 14A, the control slider 41A is driven to slide along the guiding member 14A. In other words, the control slider 41A is movable on an outer surface of the guiding member 14A. It is worth mentioning that the distance between the locking shoulder 42A and the locking groove 411A of the control slider 41A is the traveling distance of the inhaler unit 301A between the retracted position and the operation position.

The inhaler control unit 40A further comprises a slider actuator 403A extended from the control button 402A to the locker arm 401A, and a resilient element 404A coaxially mounted at the guiding member 14A for applying a pushing force against the inhaler unit 301A so as to push the inhaler unit 301A from the retracted position to the operation position, such that when the control button 402A is actuated, the locking end of the locker arm 401A is moved by the slider actuator 403A so as to disengage the locker arm 401A with the inhaler unit 301A. It is worth mentioning that the control button 402A is perpendicularly moved away from the inhaler unit 301A, i.e. the downward movement of the control button 402A, to disengage the locking end of the locker arm 401A with the locking shoulder 42A.

The inhaler control unit 40A further comprises a retraction spring 405A coupled between the slider actuator 403A and an inner wall of the hand-held housing 10′ for pushing the control button 402A toward the inhaler unit 301A after the control button 402A is pushed away from the inhaler unit 301A. While the control button 402A is pushed away the locking end of the locking arm 401A, the retraction spring 405A is compressed by the movement of the control button 402A. And, when the locking end of the locking arm 401A is disengaged with the locking shoulder 42A, a resilience force of the retraction spring 405A will push the locking arm 401A toward the inhaler unit 301A, so as to engage the hooking end of the locking arm 401A with the locking groove 411A.

Referring to FIGS. 10A and 10B of the drawings, in order to actuate the inhaler unit 301A from the retracted position to the operation position, the control button 402A is moved downwardly from the inhaler unit 301A, such that the locking end of the locking arm 401A is disengaged with the locking shoulder 42A at the inhaler unit 301A. As a result, the resilient element 404A will push the inhaler unit 301A from the retracted position to the operation position, so as to extend the inhaler unit 301A out of the hand-held housing 10′. It is worth mentioning that the control button 402A is under the tension of the retraction spring 405A, such that when the downward force at the control button 402A is released, the locking arm 401A is pushed back to its original position to engage the locking end of the locking arm 401A at the locking groove 411A so as to retain the inhaler unit 301A at the operation position.

In order to retract the inhaler unit 301A back to the retracted position from the operation, the inhaler unit 301A is pushed back into the hand-held housing 10′, such that the locking end of the locking arm 401A is slid along the surrounding wall of the inhaler unit 301A until the locking end of the locking arm 401A is re-engaged with the locking shoulder 42A, so as to lock up the inhaler unit 301A at the retracted position.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. An LED torch, comprising:
a hand-held housing having an elongated structure and defining an inhaler end, an opposed treatment end, and a treatment cavity formed at said treatment end for receiving a treatment element in said treatment cavity;
an electric power arrangement which comprises an electric power source received in said hand-held housing, an electrical-powered control unit which is electrically linked to said electric power source, and a heating arrangement provided at said treatment cavity for heating and extracting said treatment element therein;
an LED illumination unit provided at said treatment end of said hand-held housing and operatively linked to said electric power source for generating illumination light;
an inhaler arrangement which comprises a tubular inhaler unit retractably coupled at said hand-held housing and being moved between an operation position and a retracted position, wherein in said operation position, said inhaler unit is longitudinally extended out of said inhaler end of said hand-held housing to communicate with said treatment cavity for inhalation of an ingredient of said treatment element, wherein in said retracted position, said inhaler unit is longitudinally received in said hand-held housing; and
an inhaler control unit which comprises a locker arm movably provided at said held-hand housing to lock up said inhaler unit in said retracted position, and a control button being actuated to disengage said locker arm with said inhaler unit so as to enable said inhaler unit to be moved from said retracted position to said operation position.

2. The LED torch, as recited in claim 1, wherein said hand-held housing further comprises a tubular guiding member extended from said treatment cavity to said inhaler end, wherein said inhaler unit is coaxially and slidably coupled around said guiding member to move between said retracted position and said operation position.

3. The LED torch, as recited in claim 1, wherein said locker arm, which is made of bendable material, has an affixing end affixed at said hand-held housing and a hooking end engaging with said inhaler unit to lock up said inhaler unit in said retracted position, such that when said control button is moved to bend said hooking end of said locker arm outwardly, said hooking end is disengaged with said inhaler unit to enable said inhaler unit to be moved from said retracted position to said operation position.

4. The LED torch, as recited in claim 2, wherein said locker arm, which is to made of bendable material, has an affixing end affixed at said hand-held housing and a hooking end engaging with said inhaler unit to lock up said inhaler unit in said retracted position, such that when said control button is moved to bend said hooking end of said locker arm outwardly, said hooking end is disengaged with said inhaler unit to enable said inhaler unit to be moved from said retracted position to said operation position.

5. The LED torch, as recited in claim 3, wherein said inhaler control unit further comprises a control slider, having a locking groove, coupled at an inner end of said inhaler unit and slidably coupled at said guiding member, wherein said hooking end of said locking arm is engaged with said locking groove of said control slider to lock up said inhaler unit in said retracted position.

6. The LED torch, as recited in claim 4, wherein said inhaler control unit further comprises a control slider, having a locking groove, coupled at an inner end of said inhaler unit and slidably coupled at said guiding member, wherein said hooking end of said locking arm is engaged with said locking groove of said control slider to lock up said inhaler unit in said retracted position.

7. The LED torch, as recited in claim 4, wherein said inhaler control unit further comprises a slider actuator extended from said control button to align with said hooking end of said locker arm, such that when said control button is alignedly moved toward said locker arm, said hooking end of said locker arm is pushed by said slider actuator so as to bend said locker arm outwardly to disengage with said inhaler unit.

8. The LED torch, as recited in claim 6, wherein said inhaler control unit further comprises a slider actuator extended from said control button to align with said hooking end of said locker arm, such that when said control button is alignedly moved toward said locker arm, said hooking end of said locker arm is pushed by said slider actuator so as to bend said locker arm outwardly to disengage with said inhaler unit.

9. The LED torch, as recited in claim 2, wherein said inhaler control unit further comprises a resilient element coaxially mounted at said guiding member for to applying a pushing force against said inhaler unit so as to push said inhaler unit from said retracted position to said operation position.

10. The LED torch, as recited in claim 8, wherein said inhaler control unit further comprises a resilient element coaxially mounted at said guiding member for applying a pushing force against said inhaler unit so as to push said inhaler unit from said retracted position to said operation position.

11. The LED torch, as recited in claim 9, wherein said guiding member comprises an inner pipe extended from said treatment cavity and an intermediate pipe coaxially extended from said inner pipe to said inhaler end, wherein a diameter of said intermediate pipe is larger than a diameter of said inner pipe, wherein said inhaler unit is coaxially and slidably coupled within said intermediate pipe while said resilient element is coaxially coupled at said inner pipe.

12. The LED torch, as recited in claim 10, wherein said guiding member comprises an inner pipe extended from said treatment cavity and an intermediate pipe coaxially extended from said inner pipe to said inhaler end, wherein a diameter of said intermediate pipe is larger than a diameter of said inner pipe, wherein said inhaler unit is coaxially and slidably coupled within said intermediate pipe while said resilient element is coaxially coupled at said inner pipe.

13. The LED torch, as recited in claim 8, wherein said inhaler control unit further comprises a retraction spring coupled between said hand-held housing and said control button for pulling said control button after said control button is pushed toward said locking arm.

14. The LED torch, as recited in claim 12, wherein said inhaler control unit further comprises a retraction spring coupled between said hand-held housing and said control button for pulling said control button after said control button is pushed toward said locking arm.

15. The LED torch, as recited in claim 1, wherein said hand-held housing further comprises an enclosure cap pivotally coupled at said treatment end of said hand-held housing to sealedly enclose said treatment cavity, wherein said LED illumination unit comprises an LED light formed at said enclosure cap and a light switch operatively linked between said electric power source to selectively activate said LED light.

16. The LED torch, as recited in claim 14, wherein said hand-held housing further comprises an enclosure cap pivotally coupled at said treatment end of said hand-held housing to sealedly enclose said treatment cavity, wherein said LED illumination unit comprises an LED light formed at said enclosure cap and a light switch operatively linked between said electric power source to selectively activate said LED light.

17. The LED torch, as recited in claim 1, wherein said electrical-powered control unit comprises a control circuit supported in said hand-held housing for controlling an operation of said heating arrangement, an inhaler switch operatively linked to said control circuit to selectively activate said heating arrangement, and a display screen formed on said hand-held housing for displaying a status of said operation of said heating arrangement.

18. The LED torch, as recited in claim 16, wherein said electrical-powered control unit comprises a control circuit supported in said hand-held housing for controlling an operation of said heating arrangement, an inhaler switch operatively linked to said control circuit to selectively activate said heating arrangement, and a display screen formed on said hand-held housing for displaying a status of said operation of said heating arrangement.

19. The LED torch, as recited in claim 16, wherein inhaler unit is supported within said hand-held housing between said electrical-powered control unit and said electric power source, such that said electrical-powered control unit is supported above said inhaler unit while said electric power source is supported below said inhaler unit.

20. The LED torch, as recited in claim 17, wherein inhaler unit is supported within said hand-held housing between said electrical-powered control unit and said electric power source, such that said electrical-powered control unit is supported above said inhaler unit while said electric power source is supported below said inhaler unit.
